(19) United States
(12) Patent Application Publication
(10) Pub. No.: US 2021/0298608 A1
(43) Pub. Date: Sep. 30, 2021

(54) PATTERN RECOGNITION SYSTEM FOR IDENTIFYING PATIENTS WITH ISCHEMIC HEART DISEASE

(71) Applicant: Shape Medical Systems, Inc., St. Paul, MN (US)

(72) Inventors: Stephen T. ANDERSON, North Oaks, MN (US); David M. ANDERSON, White Bear Lake, MN (US); Dean J. MacCARTER, Englewood, CO (US)

(21) Appl. No.: 17/213,128

(22) Filed: Mar. 25, 2021

Related U.S. Application Data
(60) Provisional application No. 63/000,239, filed on Mar. 26, 2020.

(51) Int. Cl. A61B 5/0205; A61B 5/339; A61B 5/00; A61B 5/091; A61B 5/083; A61B 5/087; A61B 5/024; A61B 5/358; A61B 5/271

(52) U.S. Cl. CPC A61B 5/0205; A61B 5/339; A61B 5/4884; A61B 5/7275; A61B 5/091; A61B 5/271; A61B 5/087; A61B 5/024; A61B 5/0833; A61B 5/358; A61B 5/0836

(57) ABSTRACT

Systems and methods for evaluating patients for ischemic heart disease are provided. An example system includes a flow sensor to sense a respiratory flow, an analyzer to determine a respiratory gas composition of at least a portion of the respiratory flow, an ECG device configured to determine ST segment values, and a computing device. The computing device may be configured to: receive gas exchange measurements that are based on breath-by-breath data captured by the flow sensor and the analyzer during a cardiopulmonary exercise test that includes an exercise phase; receive ST segment values captured by the ECG device during the cardiopulmonary exercise test; determine an ischemic index value based on the received gas exchange measurements and ST segment values; and output the ischemic index. The ST segment values may include ST segment depression or elevation values and the ischemic index value may be determined from the ST segment depression/elevation values.

### RELATED APPLICATIONS

This application is related to U.S. Pat. No. 8,630,811, titled “Method for combining individual risk variables derived from cardiopulmonary exercise testing into a single variable” and dated Jan. 14, 2014; U.S. Pat. No. 8,775,093, titled “Pattern Recognition System for Classifying the Functional Status of Patients with Pulmonary Hypertension, Including Pulmonary Arterial and Pulmonary Vascular Hypertension” and dated Jul. 8, 2014; and U.S. Pat. No. 10,010,264, titled “Pattern recognition system for quantifying the likelihood of the contribution of multiple possible forms of chronic disease to patient reported dyspnea” and dated Jul. 3, 2018; and U.S. patent application Ser. No. 16/583,034, titled “A Pattern Recognition System For Classifying The Functional Status Of Patients With Chronic Heart, Lungs, And Pulmonary Vascular Diseases” and dated Sep. 25, 2019; each of which are hereby incorporated by reference in their entireties.

### BACKGROUND

The present disclosure relates generally to the field of medical evaluation, assessment, and/or diagnosis of patients, including symptomatic patients and patients presenting with dyspnea (also called shortness of breath) with or without fatigue, and specifically, to a process of identifying patients with ischemic heart disease.

Ischemic heart disease refers to the problems that occur in the heart due to reduction in blood supply to the heart muscle tissue. When the heart does not receive enough blood, it is deprived of necessary oxygen. Ischemic heart disease can be caused by the narrowing of the coronary arteries and is sometimes referred to as coronary heart disease (CHD) or coronary artery disease (CAD).

### SUMMARY

The present method provides an improvement in sensitivity and specificity of electrocardiogram (ECG) stress testing when compared to using classical ECG analysis alone. In addition, the present disclosure provides feedback during long-term follow-up and treatment of patients with ischemic heart diseases.

The present advance, to a large extent, obviates the current problems with existing combinations of cardio-pulmonary exercise (CPX) testing and ECG, which are discussed further herein. The present disclosure may use physiological measurements (variables) derived from serial CPX testing to track changes marking the progression and/or regression of the underlying global ischemic burden, regardless of mechanism. Exercise capacity (or cardiorespiratory fitness) is a powerful predictor of all-cause mortality. This premise appears to hold true in asymptomatic healthy individuals as well as patient populations with chronic disease. If the underlying cardiac disease process worsens, so do measures of oxygen (O2) transport, exercise capacity, and prognosis.

In accordance with the present disclosure, a new method has been found for a pattern recognition system that explains gas exchange in the lungs during exercise consisting of 1) a cardiopulmonary exercise gas exchange analyzer that gathers the observations to be classified or described, 2) a feature extraction mechanism that computes numeric information from the observations, and 3) a classification or description scheme that does the actual job of classifying or describing observations based on the extracted features.

One aspect is a system comprising: a flow sensor configured to sense a respiratory flow of the patient; an analyzer configured to determine a respiratory gas composition of at least a portion of the respiratory flow of the patient; an ECG device configured to determine an ST segment value for the patient; a computing device configured to: receive gas exchange measurements, wherein the gas exchange measurements are based on breath-by-breath data captured by the flow sensor and the analyzer during a cardiopulmonary exercise test that includes an exercise phase; receive ST segment depression values, wherein the ST segment values are captured by the ECG device during the cardiopulmonary exercise test; determine an ischemic index value based on the received gas exchange measurements and ST segment values; and output the ischemic index. The system may, for example, be for identifying patients with ischemic heart disease. The ST segment values may include changes in ST segment values, such as ST segment depression values. The ischemic index value may be determined based on the ST segment depression values.

### BRIEF DESCRIPTION OF THE DRAWINGS

FIG. 1 is a schematic drawing that illustrates the functional components of an example CPX testing system usable with the present disclosure.

FIG. 2 is a schematic drawing that illustrates an example exercise protocol that is used to place a volume load on the cardiopulmonary system.

FIG. 3 illustrates an example data table that organizes the measured data once it is acquired from the cardiopulmonary exercise gas exchange analyzer.

FIG. 4 is a flowchart of an example method that may be performed by embodiments of the system of FIG. 1.

FIG. 5 is a schematic drawing that illustrates an example breakpoint that may be identified by implementations of the method of FIG. 4.

FIG. 6 shows an example graph of ST segment measurements that may be generated by embodiments of the system of FIG. 1.

FIG. 7 illustrates example logic used to determine an ischemic index performed by embodiments of the system of FIG. 1.

FIG. 8 illustrates an example user interface that may be generated by embodiments of the system of FIG. 1.

FIG. 9 is a flowchart of an example method that may be performed by embodiments of the system of FIG. 1.

FIG. 10 illustrates an example networked CPX testing system.

FIG. 11 shows an example ECG wave.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The following detailed description, including the use of patient data, is intended to be example of a method of utilizing the concepts of the present disclosure and is not intended to be exhaustive or limiting in any manner with respect to similar methods and additional or other steps which might occur to those skilled in the art. The following description further utilizes illustrative examples, which are believed sufficient to convey an adequate understanding of the broader concepts to those skilled in the art, and exhaustive examples are believed unnecessary.

The present disclosure relates to increasing the diagnostic usefulness of ECG and cardio-pulmonary exercise test for evaluation of patient condition and specifically, for the evaluation of whether a patient suffers from cardiac ischemia. Although millions of 12 Lead ECG treadmill stress tests are performed each year to determine whether symptomatic patients have ischemic heart disease, these tests still have drawbacks regarding sensitivity and specificity for cardiac ischemia detection. Usually cardiac ischemia, if present, is triggered by unmatched increasing metabolic demand upon exertion. In addition, symptoms due to cardiac ischemia are effort dependent upon the onset of an imbalance in myocardial O₂ demand and its O₂ supply. Patients who may show significant ST depression upon exertion may not have persistent changes during recovery. If the patient does indeed undergo coronary angiography to determine the extent of the coronary lesion, coronary flow reserve may still be adequate to not warrant revascularization by percutaneous coronary intervention (PCI).

Even though stress echo can provide evidence of regional wall motion abnormalities that may occur with exertion induced ischemia, there is no gold standard functional assessment of patient functional capacity, as is provided by CPX testing.

Since electrocardiographic occurrence of abnormal ST segment depression in various ECG leads does not always agree with the Gold Standard findings of coronary angiography, the value of functional CPX testing has been proposed. Previous research by Belardinelli, et. al. (Belardinelli, Romualdo, et al. “Exercise-induced myocardial ischaemia detected by cardiopulmonary exercise testing.” European heart journal 24.14 (2003): 1304-1313) has demonstrated a significant increase in sensitivity and specificity (87%/74%) using CPX testing with ECG stress testing for ischemic heart disease detection beyond that provided by ECG stress testing alone (46%/66%), yet comparable to the detection rates by myocardial scintigraphy.

Similarly, Chaudhry and colleagues (Chaudhry, Sundeep, et al. “The utility of cardiopulmonary exercise testing to detect and track early-stage ischemic heart disease.” Mayo Clinic Proceedings. Vol. 85. No. 10. Elsevier, 2010; and Chaudhry, Sundeep, et al. “A practical clinical approach to utilize cardiopulmonary exercise testing in the evaluation and management of coronary artery disease: a primer for cardiologists.” Current opinion in cardiology 33.2 (2018): 168) have also reported the use of CPX testing for detecting ischemia induced left ventricular (LV) pump dysfunction, as observed with analysis of the O₂ pulse (O₂P) profile. In addition, they demonstrated how the flattening of the O₂P during the later phase of incremental exercise is correlated with the compensatory acceleration of heart rate relative to the work performed.

CPX testing measured oxygen pulse (VO₂/HR) or O₂P correlates very highly to direct hemodynamically measured stroke volume (SV). The CPX testing measured O₂P provides a dynamic plot with exercise time. The rate of increase of the O₂P and its attained peak during end exercise is dependent upon LV diastolic and systolic pump function and adequate coronary perfusion. It is also known that the ejection fraction (EF), which increases with exercise, usually peaks sub-maximally at the ventilatory threshold, as determined by CPX testing. Chaudhry et al. reported a linear increase of the O₂P in normal subjects with increasing work performed, as compared to a plateauing or even decreasing pattern at later phases of incremental exercise due to cardiac ischemia and pump dysfunction. As a result of a decreasing O₂P or SV, Chaudry also observed a compensatory increase in heart rate relative to watts performed as an attempt to compensate for the decreased stroke output of the heart. A reduced peak attained O₂P at end exercise was also demonstrated in patients with clinically proven coronary artery disease (CAD).

Chaudhry proposed a schematic sequence of events between cardiac muscle dysfunction to onset of ischemia and patient perception of dyspnea or chest pain/pressure. Chaudry also showed that revascularization of the “culprit” coronary vessels with significant stenosis via angiography improved the peak attained O₂P and eliminated the plateaued profile, in addition to reducing the HR to work rate slope.

The non-invasive gas exchange based pulmonary artery capacitance (GxCap) represents cardiac output or blood flow through the pulmonary circulation between the right and left heart. GxCap is dependent on right heart venous return, right ventricular (RV) pump function and the trans-pulmonary pressure gradient from the pulmonary artery (PA) to the left atrium. GxCap is comprised of two metrics: (1) end expired CO₂ partial pressure (PETCO₂) and (2) the O₂P, a close surrogate to SV, an index of ventricular contractile pump function, ventricular filling, and EF. PETCO₂ has been shown to correlate well with pulmonary blood flow at rest and during exercise, decreasing with pulmonary vasoconstriction as in the case of Pulmonary Arterial Hypertension (PAH) or being blunted in its rise during exercise due to an elevation in left atrium (LA) pressure and a reduction in the pulmonary vascular flow pressure gradient.

It is known that ischemic heart disease can increase LA pressure due to reduced pump function, stroke volume, and EF with its onset during exertion. Since both components of GxCap are known to decrease with the onset of ischemia, it is proposed that this non-invasive metric be used as a sensitive responder to pump dysfunction occurring with exertional ischemia. Graphic analysis of the dynamic GxCap plot demonstrates a clear inflection point with a subsequent downward change with ischemic burden due to both reduced cardiac stroke output (O₂P) and also reduced trans-pulmonary blood flow gradient from elevated post LA pressures. A blunted PETCO₂ rise and a reduced SV in combination increase the sensitivity to an ischemic insult resulting from reduced or inadequate coronary flow and myocardial perfusion and LV pump dysfunction.

The combination of CPX testing and 12 lead ECG has been available for over 30 years from commercial vendors. However, these “integrated systems” provide little in the way of analytic software to analyze available gas exchange variables in a way suggested by authors of various scientific papers on ischemic heart disease, including Belardinelli and Chaudhry mentioned above. These authors, however, provide only non-specific descriptions of observable events such as “plateauing” or “decreasing/increasing patterns”. This leaves the actual analysis of these events up to the pattern recognition skills of each individual interpreting physician.

In previous patent applications, the current inventors have disclosed methods for prognosis (U.S. Pat. No. 8,630,811), diagnosis (DR2) for use with patients with chronic diseases (U.S. Pat. No. 10,010,264 and U.S. patent application Ser. No. 16/583,034), each of which is incorporated by reference in its entirety. The DR2 patent, in part, describes a method of generating silos that correspond to the contribution of particular diseases or conditions to dyspnea of a patient. The present disclosure describes a method for determining the likelihood that a patient has early or advanced ischemic disease. In the context of the method described in the DR2 patent application, some embodiments of the methods provided herein describe a process for “splitting” a Heart Disease silo into two silos—one for ischemic disease and one for non-ischemic disease such as hypertensive heart disease, myocarditis, alcoholic cardiomyopathy, valvular heart disease, congenital heart disease, or cardiac dysfunction due to rapid atrial fibrillation. In some implementations, the likelihood value for a silo is defined by a scale of 0-3, where a score of 3 indicates high likelihood.

In at least some implementations of the following methods for CPX testing for ischemia detection, a linear work rate protocol is performed, either on a cycle ergometer or treadmill. For example, work rate (power) on a treadmill may be computed with a formula that uses patient weight, treadmill speed, and elevation. As another example, work rate may be calculated on a cycle ergometer based on rotations per minute and a resistance setting. In some implementations, the treadmill or cycle-ergometer may receive a target work rate for a test protocol and automatically adjust speed, elevation, or resistance to help the patient achieve the target work rate. The calculated work rate may be transmitted from a treadmill or cycle ergometer to a CPX testing system to track the work rate and the patient's adherence to the testing protocol. In some implementations, the CPX testing system may determine, based on the received work rate, when a specific phase of the test is complete and when a new phase of the test should begin. In some implementations, when the onset of cardiac ischemia is detected (or when significant cardiac ischemia is detected) in any of the ECG leads, the relative work rate and heart rate achieved at that point in the CPX test may be recorded.

General Considerations—Embodiments of the present disclosure include a pattern recognition system consisting of a) a cardiopulmonary exercise gas exchange analyzer and a 12 lead ECG that gathers the observations to be classified or described, b) a feature extraction mechanism that computes numeric information from the observations, and c) a classification or description scheme that classifies or describes observations based on the extracted features.

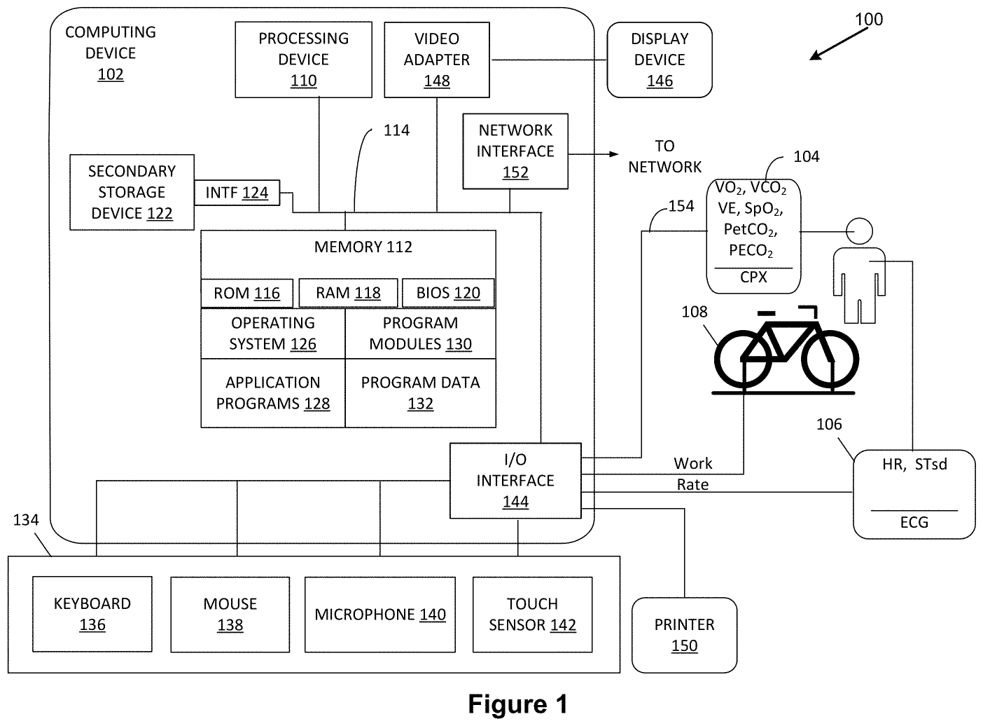

Equipment—With this in mind, an example system is shown in FIG. 1, which illustrates an example CPX testing system 100, whereby a CPX test may be conducted and the results displayed in accordance with the method of the present disclosure. In this example, the system includes a computing device 102, a gas exchange analyzer 104, an ECG device 106, and exercise equipment 108. The computing device 102 illustrated in FIG. 1 can be used to execute the operating system, application programs, and software modules described herein.

The computing device 102 includes, in some embodiments, at least one processing device 110, such as a central processing unit (CPU). A variety of processing devices are available from a variety of manufacturers, for example, Intel or Advanced Micro Devices. In this example, the computing device 102 also includes a system memory 112, and a system bus 114 that couples various system components including the system memory 112 to the processing device 110. The system bus 114 is one of any number of types of bus structures including a memory bus, or memory controller; a peripheral bus; and a local bus using any of a variety of bus architectures.

Examples of computing devices suitable for the computing device 102 include a desktop computer, a laptop computer, a tablet computer, a mobile computing device (such as a smartphone, an iPod® or iPad® mobile digital device, or other mobile devices), or other devices configured to process digital instructions.

The system memory 112 includes read only memory 116 and random-access memory 118. A basic input/output system 120 containing the basic routines that act to transfer information within computing device 102, such as during start up, is typically stored in the read only memory 116.

The computing device 102 also includes a secondary storage device 122 in some embodiments, such as a hard disk drive, for storing digital data. The secondary storage device 122 is connected to the system bus 114 by a secondary storage interface 124. The secondary storage devices 122 and their associated computer readable media provide nonvolatile storage of computer readable instructions (including application programs and program modules), data structures, and other data for the computing device 102.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random-access memories, or read-only memories. Some embodiments include non-transitory computer-readable media. Additionally, such computer readable storage media can include local storage or cloud-based storage.

A number of program modules can be stored in secondary storage device 122 or system memory 112, including an operating system 126, one or more application programs 128, other program modules 130 (such as the software engines described herein), and program data 132. The computing device 102 can utilize any suitable operating system, such as Microsoft Windows™, Google Chrome™ OS or Android, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 102 through one or more input devices 134. Examples of input devices 134 include a keyboard 136, mouse 138, microphone 140, and touch sensor 142 (such as a touchpad or touch sensitive display device). Other embodiments include other input devices 134. The input devices 134 are often connected to the processing device 110 through an input/output interface 144 that is coupled to the system bus 114. These input devices 134 can be connected by any number of input/output interfaces, such as a parallel port, serial port, game port, or a universal serial bus. Wireless communication between input devices and the interface 144 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 146, such as a monitor, liquid crystal display device, projector, or touch sensitive display device, is also connected to the system bus 114 via an interface, such as a video adapter 148. In addition to the display device 146, the computing device 102 can include various other peripheral devices, such as a printer 150 for printing reports or speakers (not shown) for providing audible feedback or instructions during CPX tests.

When used in a local area networking environment or a wide area networking environment (such as the Internet), the computing device 102 is typically connected to the network through a network interface 152, such as an Ethernet interface or WiFi interface. Other possible embodiments use other communication devices. For example, some embodiments of the computing device 102 include a modem for communicating across the network.

The computing device 102 typically includes at least some form of computer readable media. Computer readable media includes any available media that can be accessed by the computing device 102. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random-access memory, read-only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 102.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device 102 illustrated in FIG. 1 is also an example of programmable electronics, which may include one or more such computing devices, and when multiple computing devices are included, such computing devices can be coupled together with a suitable data communication network so as to collectively perform the various functions, methods, or operations disclosed herein.

In some implementations, the gas exchange analyzer 104 includes a carbon dioxide sensor that can determine a concentration of carbon dioxide in a gas sample. Some implementations also include an oxygen sensor that can determine a concentration of oxygen in a gas sample. Some implementations do not include an oxygen sensor. The gas exchange analyzer 104 may also include a flow volume sensor that can determine a flow volume of gas exhaled or inhaled by the patient. For example, the flow volume sensor may include one or more pressure transducers. The pressure transducers may determine a change in pressure as the gas passes through an orifice in a tube (pneumotach) through which the patient is breathing. Based on the change in pressure, the flow volume may be determined for the gas being exhaled and inhaled by the patient. These flow measurements may be combined with one or more of the CO₂ or O₂ measurements from the corresponding sensors to determine various physiological measurements (or variables) of the patient as described further herein. For example, the gas exchange analyzer 104 may determine breath-by-breath values of VO₂, VCO₂, VE, PetCO₂, and PECO₂.

In some implementations, the gas exchange analyzer 104 also includes a pulse oximetry device that measures peripheral capillary oxygen saturation (SpO₂). Various types of pulse oximetry devices may be included such as fingertip, earlobe, temporal, or nasal pulse oximetry devices.

The ECG device 106 may include an ECG, such as a 12-lead ECG. In at least some implementations, the ECG device 106 may interface with the patient to record electrical activity of the patient's heart. For example, multiple leads (i.e., electrodes) may be placed on the patient's skin at various locations, such as on the patient's chest and limbs. The ECG device 106 may then determine electrical changes that accompany the cardiac cycle. The ECG device 106 may record these electrical changes as waveforms.

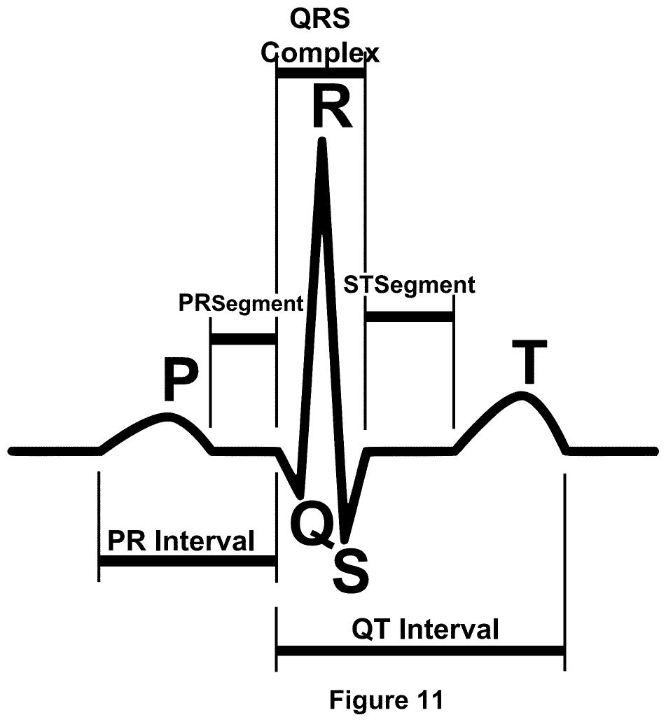

The ECG device 106 may also determine various properties of the waveforms, such as a magnitude of an ST segment depression. For example, ST segment depression may refer to the amount by which the ST segment of an ECG waveform drops below a baseline level. The baseline is the isoelectric line in which there is no positive or negative electrical charges. An example ECG wave is shown in FIG. 11. The ECG wave includes three main components: the P wave, which represents the depolarization of the atria; the QRS complex, which represents the depolarization of the ventricles; and the T wave, which represents the repolarization of the ventricles. The P wave is connected to the QRS complex by the PR segment, and the QRS complex is connected to the T wave by the ST segment. The ECG device 106 may also determine a heart rate (e.g., beats per minute (BPM)) for the patient based on the measurements from one or more of the leads.

Although most of the examples included herein describe systems the include an ECG, at least some implementations do not include an ECG device. In these implementations, the strength of evidence for the patient suffering from cardiac ischemia (or the strength of evidence that the cardiac ischemia is contributing to the patient's dyspnea) is determined based on gas exchange measurements entirely without evaluating ST segment elevation or depression from an ECG device.

In this example, the exercise equipment 108 is a cycle ergometer. In some implementations, the exercise equipment 108 may include a simple stair step of a known height or any other exercise modality such as a treadmill or hand ergometer. A gas exchange analyzer 104 interfaces with the subject (the subject is also referred to as a patient) during operation of the exercise test. Various physiological variables may be determined for the patient based at least in part on measurements by the system 100 during a CPX test, such as a submaximal CPX test. The physiological variables may be selected from heart rate (HR), ventilation (VE), rate of oxygen uptake or consumption (VO₂) and carbon dioxide production (VCO₂), end tidal CO₂ (PetCO₂), mixed expired CO₂ (PECO₂), or other variables derived from these basic measurements. In some implementations, a respiratory exchange ratio (RER) is calculated by dividing the measured VCO₂ value by the measured VO₂ value. Physiological data collected is fed into the computing device 102 via a conductor 154, or other communication device.

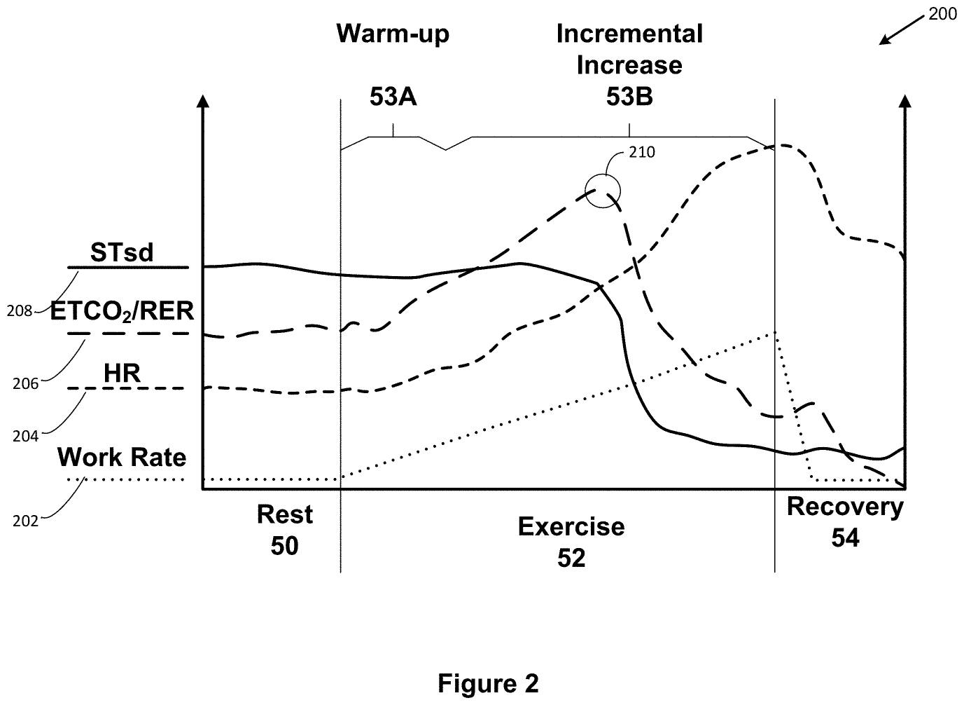

Data Gathering: FIG. 2 shows an example graph 200 of an embodiment of a CPX protocol and example data gathered as a test subject performs the protocol. The CPX protocol is divided in to a rest phase 50, an exercise phase 52, and a recovery phase 54. The rest phase 50 may include a time period during which the test subject is monitored without performing any exercise activity. For example, the test subject may be instructed to sit during the rest phase 50 and engage in no or minimal movement. The measurements gathered during the rest phase 50 may establish a baseline level for the test subject. Although alternatives are possible, the rest phase 50 may last for 1 minute.

The exercise phase 52 may collect data indicative of the patient's response to increased exercise workload. In this example, the exercise phase 52 includes a warm-up subphase 53A and an incremental increase subphase 53B. The warm-up subphase 53A may correspond to a time period at the beginning of the exercise phase 52 in which the test subject performs the exercise activity at a constant rate with little to no resistance to get used to performing the exercise activity. For example, for test protocols that involve using a cycle ergometer, the warm-up phase may involve free wheeling while the cycle ergometer is unloaded (e.g., the cycle ergometer provides no resistance). Although alternatives are possible, the warm-up subphase 53A may last for between 15 second and 2 minutes.

During the incremental increase subphase 53B, the workload of the exercise activity is increased. The increase may be a gradual increase or stepwise increase. For example, in embodiments that include a cycle ergometer, the computing device 102 may transmit instructions to the cycle ergometer to increase the work rate for the test subject (e.g., by increasing resistance). Implementations that include other exercise modalities such as a hand ergometer or treadmill may operate similarly. Implementations that use a step may increase the workload by instructing the test subject to increase stepping cadence by, for example, providing audible prompts such as producing a metronome/beat sound at the target cadence. Although alternatives are possible, the incremental increase subphase 53B may last for 3 minutes. In some implementations, the incremental increase subphase 53B may last for between 90 seconds and 5 minutes.

The recovery phase 54 may include a time period where the test subject is monitored after completion of the exercise phase 52. In some implementations, the test subject does not perform any additional exercise activity during the recovery phase 54. In some implementations, the test subject may perform exercise in a decreasing intensity that reduces to no exercise. During the recovery phase 54, measurements may be gathered that correspond to the test subject's recovery from the demands of exercise. In some implementations, all physiologic measurements are gathered during the recovery phase 54. In some implementations, only ECG measurements may be gathered during a portion of the recovery period (e.g., to continue monitoring the patient for safety purposes). Although alternatives are possible, in some implementations the recovery phase 54 may last for 2 minutes.

In some implementations, the test protocol automatically moves from one phase to another. In some implementations, a test administrator may trigger the end of one phase and the beginning of another. In some implementations, a user interface may be generated that indicates when specific physiological metrics have been achieved and prompts the test administrator to start a next phase of the test protocol. The data measured during exercise quantifies how the test subject is able to function in the physical world in terms of the physiologic changes that the test subject experiences when engaged in the performance of daily physical work.

The physiologic changes in the test subject may be measured using the gas exchange analyzer 104 to measure selected variables associated with one or more of oxygen consumption (VO₂), carbon dioxide production (VCO₂), end tidal CO₂ (ETCO₂), mixed expired CO₂ (PECO₂), SpO₂ and, respiratory exchange ratio (RER), and the ECG device 106 to measure heart rate (HR) and ST segment depression (STsd).

As the test is being performed, various physiological measurements (physiological variables) may be plotted on the graph 200. For example, the graph 200 includes a work rate plot 202, a heart rate plot 204, an ETCO₂/RER plot 206, and a ST segment depression (STsd) plot 208.

The work rate may also be referred to as power. In some implementations, the work rate is measured in watts. As can be seen in this example, after the rest phase 50, the work rate increases gradually throughout the exercise phase 52, and then decreases rapidly during the recovery phase 54. Although alternatives are possible, the work rate is shown as increasing at a uniform rate throughout the exercise phase 52 in the work rate plot 202.

The HR plot 204 represents the patient's heart rate as the test protocol is completed. In some implementations, the HR is determined using the ECG device 106. The HR may also be determined using a pulse oximeter device of the gas exchange analyzer 104. As can be seen in FIG. 2, the HR is influenced by the work rate. In some implementations, the graph 200 may include an upper HR band. The position of the upper HR band may be determined based on a maximum predicted heart rate (e.g., based on age) for the test subject. For example, the HR band may be positioned at 85% of the max predicted HR for the test subject. As another example, the HR band may be positioned at 75% of the max predicted HR for the test subject when the test subject has been administered beta blocker medication. In some implementations, the system 100 may stop, alter, or prompt the test administrator to stop or alter the test protocol when it is determined that the patient's HR has exceeded the HR band. For example, the system 100 may generate an alert on a user interface indicating that the exercise phase 52 of the test should continue for a limited time period such as one minute after the test subject's HR crosses into the HR band.

The ETCO₂/RER plot 206 represents the result of dividing the end tidal CO₂ value by the respiratory exchange ratio. In some implementations, a breakpoint 210 is identified in the ETCO₂/RER plot 206. The breakpoint 210 may correspond to a point in the plot 206 where the plot changes from increasing to decreasing while the work rate plot 202 continues increasing. Various example methods for identifying the breakpoint are discussed herein. In some implementations, the system 100 may alter or prompt a caregiver to alter the test protocol based on identifying the breakpoint. For example, the system 100, may prompt the caregiver to continue the test for a limited time period or to end the test after identifying the breakpoint.

The STsd plot 208 represents the ST segment depression as determined by the ECG device 106. In some implementations, lower values on the STsd plot 208 indicate an increase in STsd (i.e., the segment depression has become more negative). Reductions in STsd during exercise may be an indicator of ischemic heart disease. In some implementations, the graph 200 may include a lower STsd threshold (or band). For example, the lower STsd band may correspond to a STsd of −1 mm or 0.1 mv. In some implementations, the system 100 may stop, alter, or prompt the user to stop or alter the test protocol when it is determined that the patient's STsd has reached the lower threshold. For example, the system 100 may generate an alert on a user interface indicating that the exercise phase 52 of the test should continue for a limited time period such as one minute after the test subject's STsd reaches the lower threshold.

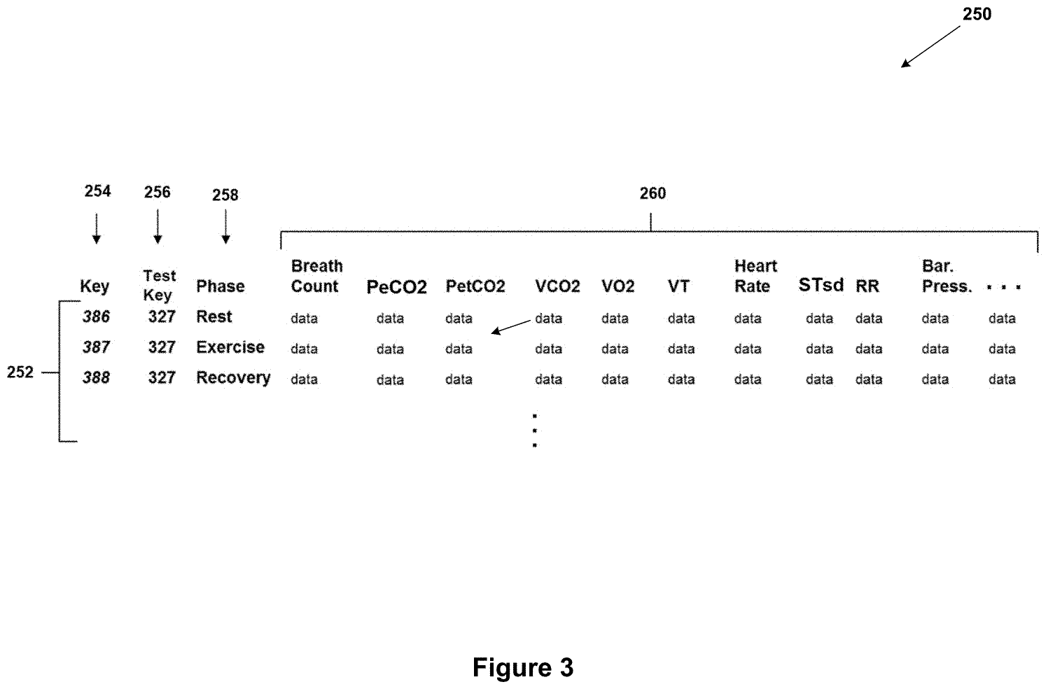

By means of aspects of the feature extraction mechanism, classification and quantification schemes, the present disclosure enables an observer to gain new and valuable insight into the present condition and condition trends in patients. Thus, in accordance with at least some embodiments, a cardiopulmonary exercise gas exchange analysis is completed for each breath-by-breath data set (FIG. 3).

Data acquired by the CPX testing system 100 may be stored in a relational database or another data store as illustrated in FIG. 3. The relational database may be stored in the memory 112, on the secondary storage device 122, or elsewhere such as on a remote computing system. In some implementations, data for each patient and each test is stored into separate subsets of data representing the rest phase 50, the exercise phase 52, and the recovery phase 54 for use by the feature extraction mechanism.

An example of one embodiment of a relational database table 250 for storing data acquired by the gas exchange testing system 100 is illustrated in FIG. 3. The table 250 contains a plurality of rows 252, each of which represents data captured during a breath of the gas exchange test. Each of the rows 252 includes a key 254, a test key 256, a phase 258, and measurement data 260.

The key 254 is used by other tables in the database to reference a particular breath/row. The test key 256 is a foreign key to row in a test table (not shown) in the database. Using the test key 256, each breath/row is associated with a test. The phase 258 associates the breaths/rows of data for each patient and each test with the appropriate phase (e.g., rest, exercise, or recovery) for use in feature extraction and classification.

Additionally, the table 250 includes multiple columns of measurement data 260. In the example shown, the measurement data includes a breath count, PeCO₂, PetCO₂, VCO₂, VO₂, VT, Heart Rate, STsd, Respiratory Rate, and Barometric Pressure. In other embodiments, more, fewer, or different measurements are stored in each row.

### Example Feature Extraction Steps

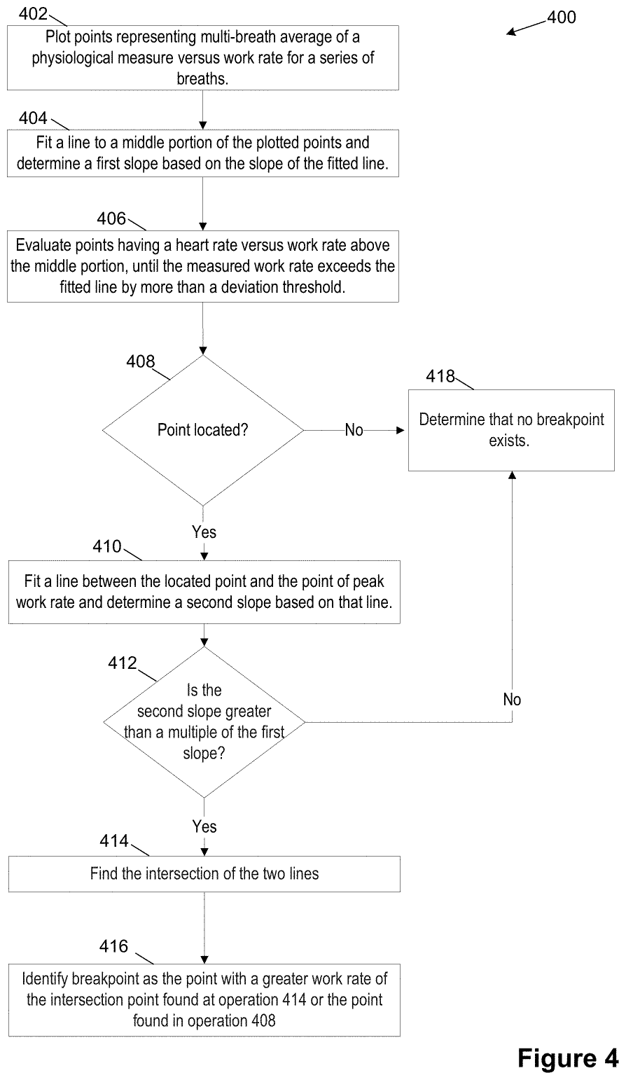

FIG. 4 is a flowchart of an example method 400 for determining whether a breakpoint (or inflection point) is present in a sequence of physiological measurements during a CPX test. The breakpoint may represent a change in the slope of physiological measurement or ratio of multiple physiological measurements during the exercise phase of a CPX test. In some implementations, the breakpoint is identified when the magnitude of the change in slope exceeds a specific threshold. Depending on the physiological measure, the breakpoint may be identified as a positive change (non-linear increase in response) or a negative change (a blunted response).

In some implementations, the breakpoint is identified within a graph of a physiological measurement or a ratio of a physiological measure, such as graphs of HR versus work rate, VO₂ versus work rate, and GxCap versus work rate. In some implementations, the presence of such a breakpoint is considered as evidence of myocardial ischemia. The breakpoint may refer to the point during exercise at which HR, VO₂, GxCap, or O₂ pulse starts to increase at a slower rate (or faster rate) than work rate or may actually decline.

The following examples will discuss HR versus work rate, but it should be understood that the following analysis may be performed in the same or a similar manner for other physiological measurements versus work rate, such as VO₂ versus work rate and GxCap versus work rate.

At operation 402, points representing multi-breath averages of a physiological measurement versus work rate (watts) are plotted for a series of breaths. For example, the multi-breath average HR versus work rate can be calculated for a plurality of breaths that have been captured sequentially by the system 100. The plurality of breaths may contain a predetermined number of breaths such as eight breaths, although other numbers of breaths can be used. In some embodiments, the HR and work rate values for the breaths are determined using the system 100.

Next, at operation 404, a line is fit to a middle portion of the points plotted in operation 402. For example, the middle portion may be identified by selecting points having a heart rate (or other physiological measure) versus work rate value between a lower threshold value and an upper threshold value. In some embodiments, the lower threshold value is 25% of the peak heart rate versus work rate value and the upper threshold value is 75% of the peak heart rate versus work rate value. However, other embodiments identify the middle portion based on other upper and lower threshold values (e.g., 20-80%, 30-70%, 40-60%, 40-75%). Although alternatives are possible, least squares linear regression is used to fit a line to the middle portion of the plotted points in this example. A first slope is then determined as the slope of the fitted line.

At operation 406, a point in which the heart rate versus work rate value exceeds the fitted line by more than a deviation threshold is identified, if one exists. In some implementations; the plotted points from operation 402 are evaluated. In some implementations, individual breaths are evaluated. For example, each breath (or a sample of breaths) may be examined starting above the middle portion (e.g., breaths having a heart rate versus work rate value exceeding the upper threshold value used in operation 404 are examined in order of increase heart rate versus work rate value starting from the lowest breath having the lowest heart rate versus work rate value; or breaths occurring after (later in the CPX test than) the identified middle portion), until the heart rate versus work rate value exceeds the fitted line by a set amount (i.e., a deviation threshold), such as 10 percent. Other values can be used for the deviation threshold such as values selected from the range 8-12%, 5-15%; 10-20%; or other ranges. Although this example describes identifying the breakpoint based on exceeding the fitted line, it should be understood that in some physiological measurements a breakpoint is identified based on deviating below the fitted line by at least the deviation threshold (i.e., for some physiological measurements breakpoints are identified based on a decrease in the slope rather than an increase).

A determination whether or not such a point exists is made in operation 408. If so, control is passed to operation 410. Otherwise, control is passed to operation 418.

In operation 410, a second line is defined between the point located at operation 408 and the point of peak heart rate versus work rate. Additionally; a second slope is determined from the second line.

Next, at operation 412, the second slope is compared to the first slope. If the second slope is equal to or greater than a predetermined multiple of the first slope, then control is passed to operation 414, Although alternatives are possible, the predetermined multiple is 1.5 in at least some embodiments. Otherwise, if the second slope does not equal or exceed the predetermined multiple of the first slope, control is passed to operation 418.

At operation 414, an intersection point of the two lines is determined.

Next, at operation 416, the breakpoint is calculated as the greater of the heart rate versus work rate value at the intersection point found in operation 414 or the heart rate versus work rate value of the point found in operation 408.

As noted, if no point is located in operation 408 or the second slope is not greater than the predetermined multiple of the first slope as noted in operation 412, control is passed to operation 418, where it is determined that no breakpoint has been found.

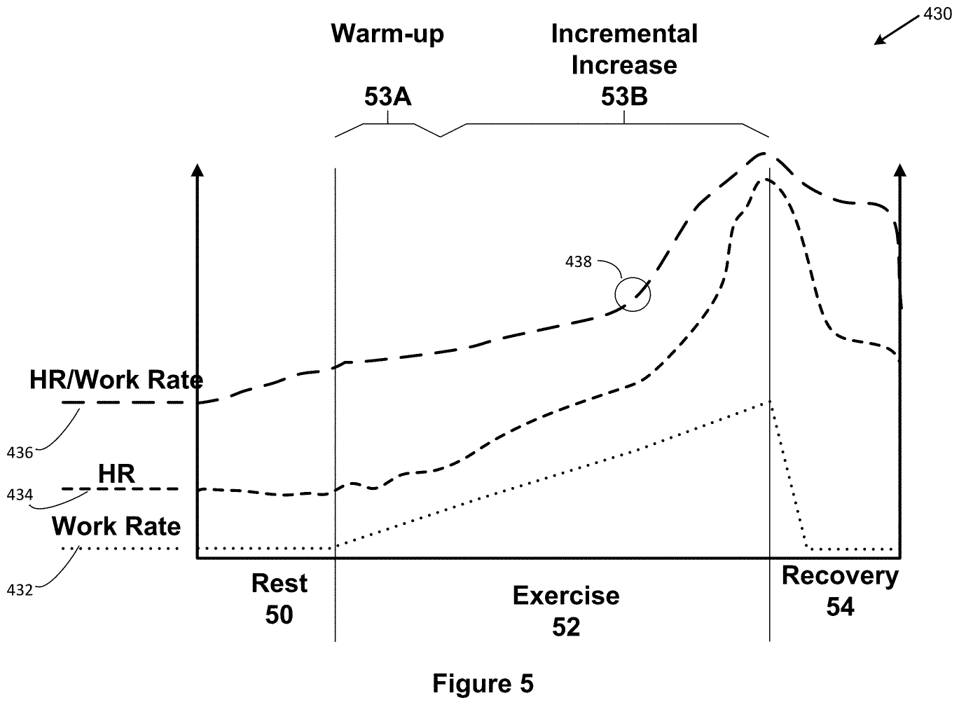

An example graph 430 showing a breakpoint 438 in a heart rate versus work rate plot 436 is shown in FIG. 5. The graph 430 includes a work rate plot 432, a heart rate (HR) plot 434, and the heart rate versus work rate (HR/Work Rate) plot 436. In this example, the breakpoint 438 may be the point in the heart rate versus work rate plot found at operation 406 of the method 400 described above. This is one example of such an analysis.

The CPX protocol shown in FIG. 5 is similar to the CPX protocol shown in FIG. 2 and includes the rest phase 50, the exercise phase 52, and the recovery phase 54. The exercise phase 52 includes the warm-up subphase 53A and the incremental increase subphase 53B. These phases and subphases have been described previously with respect to at least FIG. 2.

In some implementations, the method 400 is automated so that the computing device 102 is programmed to perform the steps of the method 400 in a semi- or fully-automated manner. This allows the computing device 102 to calculate the breakpoint automatically with little or no input from the caregiver. For example, the method 400 may allow a computing device to identify the breakpoint without input from a user which may not otherwise be possible with conventional techniques and equipment.

Once the existence or lack thereof of the breakpoint has been confirmed, the physiological measurements can be used to evaluate the likelihood of myocardial ischemia for the patient.

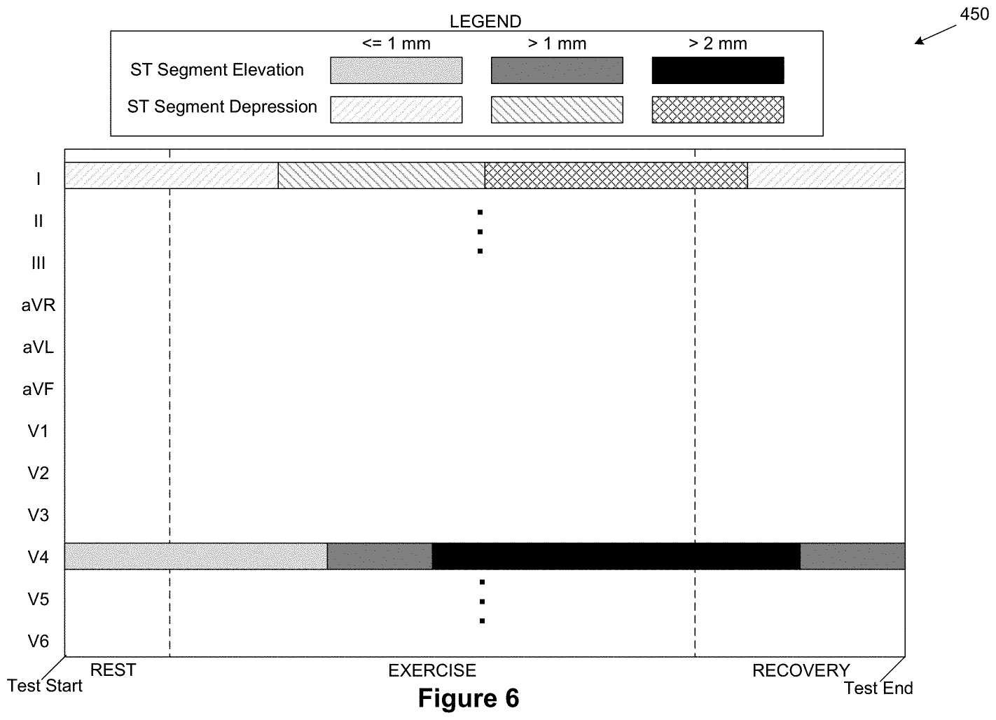

FIG. 6 shows an example graph 450 of ST segment measurements captured by some embodiments during an example CPX protocol as a test subject performs the protocol. In some implementations, the system 100 generates a user interface that includes a graph similar to the graph 450.

In this example, time is shown on a horizontal axis. Here, the time is divided into phases of the CPX test. In this example, the test includes a rest phase, an exercise phase, and a recovery phase. Although not shown in this figure, any of the phases may be divided into subphases. For example, the exercise phase may be divided into a warm-up subphase and an incremental exercise subphase. In some implementations, the warm-up subphase may be labeled “freewheel” when a cycle ergometer is being used (e.g., the wheel of the cycle ergometer spins freely without providing any resistance to the test subject). The units of time and duration of the test is not shown on this figure. The CPX test can run for any length of time. For example, the CPX test may be a 6-minute test or a longer test.

The vertical axis corresponds to the ECG leads. Each row may correspond to a specific ECG lead. In this example, labels for 12 ECG leads are shown. However, data is only shown for two of the leads (I and V4) to simplify the graph. In this example, ST segment amplitude in calibrated mm or my for ECG leads is presented. This data may be gathered by the ECG device 106. The rows for specific ECG leads are shaded to indicate the measured ST segment values throughout the duration of the test.

In this example, the graph 450 includes a legend that indicates how the measurements from the ECG leads are shaded. In this example, darker shading is indicative of a greater magnitude, solid shading is used to indicate ST segment elevation (STse), and cross-hatched shading is used to indicate ST segment depression (STse). Some implementations may shade ECG measurements differently using color other indicators of ST segment values. Gradients may be used to indicate colors that represent intermediate values. In some implementations, on the leads that meet specific criteria are displayed. For example, any of the 12 leads that include an ST segment elevation or depression that exceeds a specific threshold value are displayed. ECG leads that do not meet threshold are not displayed. In some implementations, ECG leads that meet the criteria are displayed with highlighting while those that do not are displayed without highlighting. Examples of displaying leads or portions of leads on the graph 450 with or without highlighting are provided below.

Although the graph 450 is shown as a 2D graph with time on the horizontal axis, ECG leads on the vertical axis, and ST segment values indicated with shading, other embodiments are possible too. For example, some implementations may use 3D graphs. In some implementations, different thresholds may be used to determine colors for different leads. For example, an STsd of −1 mm in a limb lead may be shown using the same color as an STsd of −2 mm in a precordial lead.

Some implementations include a user interface that allows a caregiver to identify leads or segments of measurements from a lead that are indicative of high levels of STsd (or where the ST segment is otherwise visually significant). The user interface may allow the caregiver to view more information about the signals from the ECG leads when a caregiver selects a lead or a segment of a lead. For example, in response to a caregiver selecting (e.g., clicking on) a specific time along a row of a lead, the corresponding ECG trace for the selected lead during that specific time may be displayed on the user interface. In some implementations, the ECG traces for multiple or all leads may be displayed for the specific time on the user interface.

In some implementations, the system may automatically identify or suggest leads or segments of measurements from a lead that are indicative of high levels of STsd. Based on the leads identified, the system may graphically or textually indicate arteries or regions of the heart that are likely locations of possible cardiac ischemia. For example, the identified locations of possible cardiac ischemia may include lateral, inferior, or anterior locations. The identified locations of possible cardiac ischemia may also include suspect obstructed/diseased coronary vessels such as left anterior descending (LAD), circumflex, or right coronary vessels. Some implementations may include a degree or magnitude of ischemia for the identified arteries or regions. The number of leads that provide evidence of significance may correlate to the number of coronary vessels with disease.

In some implementations, the locations of possible cardiac ischemia are determined based on the identified leads using a table that maps leads with significant ST segment changes to locations of possible cardiac ischemia such as the following:

| Leads with | Possible | Possible |
|---|---|---|
| significant ST | affected | Occluded |
| segment changes | myocardial | Coronary |
| (↓or↑) | area/location | Artery |
| V1-V2 | septal | Proximal LAD |
| V3-V4 | anterior | LAD |
| V5-V6 | apical | Distal LAD, LCx |
| | | or RCA |
| I, aVL | lateral | LCx |
| II, aVF, III | inferior | 90% RCA, 10% LCx |

Some implementations use different tables or different methods to suggest locations of possible cardiac ischemia. A caregiver may then review the suggestions and determine how to proceed with treatment and diagnosis of the test subject.

In some implementations, the system 100 may cause one or more regions of the graph 450 to be displayed differently based on gas exchange measurements. For example, a region of the graph 450 may be highlighted based on where a breakpoint was detected within a test. For example, the portions of some or all of the ECG lead traces that occurred simultaneously with the breakpoint may be highlighted. The breakpoint may be identified from various physiological measurements or ratios of physiological measurements. For example, the time of a breakpoint may be identified in the ratio of HR/WR, VO2/WR, or GxCAP/WR. In some implementations, a breakpoint is identified based on ETCO2/HR/WR. The time of a breakpoint may refer to the time within the test that the breakpoint was observed within the physiological measurements.

In some implementations, a portion extending a specific duration of time after the breakpoint is highlighted. A portion extending a specific duration of time before the breakpoint may also be highlighted. In some implementations, the duration of highlighted time before and after the breakpoint is the same, in some the durations are different. In some implementation, only a portion after the identified breakpoint is highlighted. In some implementations, a portion of the exercise phase may be highlighted from the time at which the breakpoint occurred through to the end of the exercise phase.

As used herein, highlighting refers to displaying in a manner as to draw a viewer's attention. Various implementations use various techniques to highlight regions of the graph 450. Highlighting a region of the graph includes displaying the highlighted portion differently than unhighlighted portions. For example, the highlighted portion may be displayed in color, while the highlighted portion may be in grayscale. The highlighted portion may be displayed using a specific color or set of colors that are different than the colors used to display the unhighlighted regions. In some implementations, the highlighted regions are displayed more brightly than unhighlighted regions. The highlighted regions may be outlined, circled, or may be displayed with a border that is not displayed on unhighlighted regions.

In some implementations, the graph 450 may include one or more vertical lines that indicate when during a test a breakpoint was detected in the gas exchange measurements. Some implementations also overlay a plot of one or more physiological measurements or ratios of physiological measurements over the rows corresponding to ECG leads. For example, some implementations may overlay O2 uptake on the graph 450.

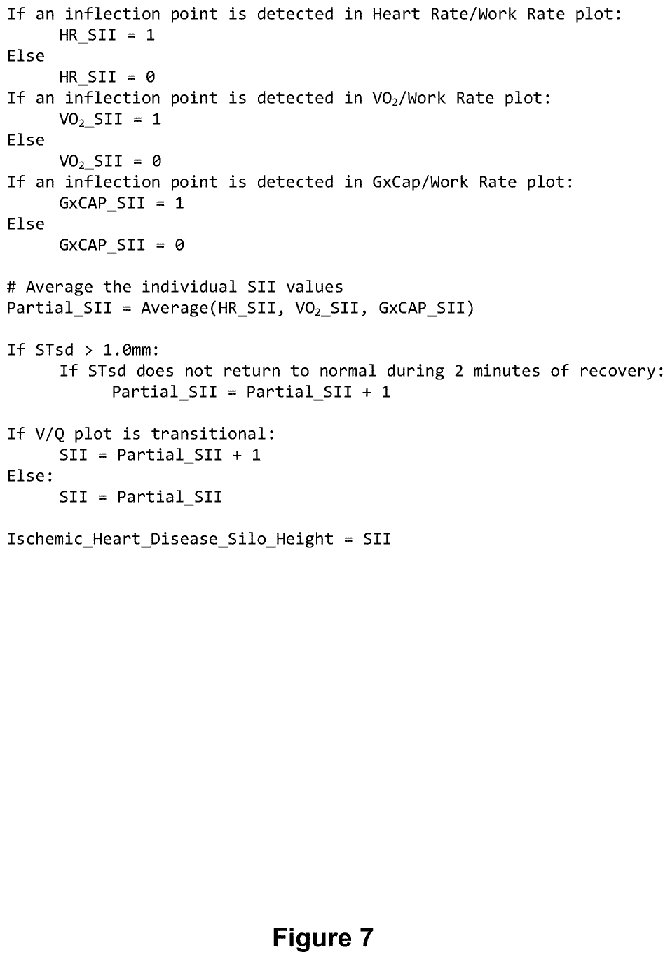

FIG. 7 illustrates example logic used to determine an ischemic index (sometimes referred to as a Shape Ischemia Index (SII)) value in at least some embodiments. The ischemic index value may indicate a likelihood that the patient suffers from cardiac ischemia. For example, higher ischemic index values may indicate a higher likelihood that the patient suffers from cardiac ischemia than lower ischemic index values. The ischemic index value may indicate the strength of evidence that the patient is suffering from cardiac ischemia. In some implementations, higher ischemia index values may indicate that further diagnostic tests such angiography or therapeutic interventions are recommended. The SII value may be used as an ischemic heart disease silo score in some implementations, which may be presented graphically or otherwise.

As another example, the SII may be computed as follows:

Step 1—The first step involves a determination of the presence or lack of presence of a “breakpoint”, which may be identified as a measurable change in a line of regression fitted to a variable pair which begins at the start of exercise and ends at the end of exercise.

Step 2—For one or more pairs of variables, provide a breakpoint score of 1 for each pair if a breakpoint is detected and a breakpoint score of 0 if not. In some implementations, at least 3 pairs of variables are used. For example, the 3 variable pairs may be HR and WR (or HR/WR); VO2 and WR (or VO2/WR); and GxCap and WR (or GxCap/WR). The breakpoint scores from these pairs may then be combined. For example, these breakpoint scores may be averaged (e.g., by summing the breakpoint scores and dividing by the number of breakpoint scores) to determine a partial SII score. In some implementations, additional variable pairs are included. For example, other variable pairs included in some implementations include ETCO2/HR and WR (ETCO2/HR/WR); GxCAP/HR and WR (GxCAP/HR/WR); O2Pulse/respiratory rate (RR) and WR (O2Pulse/RR/WR). In some implementations, the breakpoint scores may be combined by summing, weighted averaging, or otherwise. The combining may include scaling the combined breakpoint scores to a specific range. For example, the breakpoint scores may be summed and scaled to a range between 0 and 1.

Although step 2 describes providing a score of 1 based on the presence or absence of a breakpoint, other embodiments may provide different scores. Some embodiments may provide a score that is determined based not only on whether a breakpoint is detected but also when. A higher score may be provided based on, for example, when during the test the breakpoint (or breakpoints) is detected (e.g., a score of between 0 and 1 may be provided based on the percentage of the test completed before the breakpoint).

In some implementations, when a breakpoint is detected using any of the methods described herein, a HR for the patient is also determined and recorded. This HR may, for example, indicate a lower bound of a functional insult zone (or functional impact zone) for the patient. The breakpoint may occur in any of the physiological measurements or pairs of variables that have been previously described, such as HR and WR (or HR/WR); VO2 and WR (or VO2/WR); GxCap and WR (or GxCap/WR), ETCO2/HR and WR (ETCO2/HR/WR); GxCAP/HR and WR (GxCAP/HR/WR); O2Pulse/respiratory rate (RR) and WR (O2Pulse/RR/WR). This heart rate may be recorded on a computer readable media. During follow-up CPX testing, the HR at the lower bound of the functional insult zone may be re-calculated and used as an indicator of disease progression and therapy effectiveness for the patient. For example, if the patient the HR at the lower bound of the functional insult zone increase, the CPX test may indicate that the patient has improved (e.g., due to a therapy applied between CPX tests). This HR for the functional insult zone may be monitored repeatedly as the patient undergoes various therapeutic intervention to treat cardiac ischemia.

Step 3—If the ST segment depression at the end of exercise meets a specific criteria, the partial SII score is increased. For example, if the ST segment depression is >1 mm, the partial SII score may be increased by 1 (i.e., add 1 to the partial SII Score), otherwise the partial SII score is not altered at this step. In some implementations, the partial SII score is incremented based on the occurrence of ST segment depression in a single lead. In some implementations, the partial SII score is incremented if ST segment depression occurs in specific leads or at least a specific number of leads. In some implementations, the partial SII score is incremented non-uniformly based on the number of leads in which (and in some cases in which specific leads) ST segment depression occurs.

In some implementations, the partial SII score may be increased by a different amount than 1. For example, the partial SII score may be increased non-uniformly based on the duration of time between one or more of the breakpoints discussed with respect to step 2 and the occurrence of the ST segment depression. For example, if the breakpoint occurs before the ST segment depression, the SII score may be increased by a larger value than if the breakpoint occurs after the ST segment depression.

Step 4—In some implementations, if the partial SII score was increased in step 3 based on the ST segment depression at the end of exercising meeting a specific criteria, the partial SII score may be reduced based on the ST segment depression returning to normal during 2 minutes of recovery (e.g., the ST segment depression no longer meets the criteria used in step 3. For example, the partial SII score may be reduced by 1 if it was increased in step 3 and the ST segment depression returns to normal during 2 minutes of recovery, otherwise the partial SII score is not reduced.

Step 5—If the VQ plot (as described in U.S. patent application Ser. No. 16/583,034, titled “A Pattern Recognition System For Classifying The Functional Status Of Patients With Chronic Heart, Lungs, And Pulmonary Vascular Diseases” and dated Sep. 25, 2019) results in a ventilation/perfusion vector with a midpoint located in the LV Dysfunction or Transitional zone, add 1 to the partial SII score.

In some implementations, ventilation/perfusion vector is plotted from the gas exchange measurements. The vector may include (or start at) a first point based on a rest value. A first coordinate value of the first point may be based on mixed expired CO2 (PECO2) and a second coordinate value of the first point may be based on end tidal CO2 (PetCO2). The physiological measurements for the first point may be captured at the end of a rest phase of a CPX test.

The vector may include (or end at) a second point based on an exercise value. A first coordinate value of the second point may be based on mixed expired CO2 (PECO2) and a second coordinate value of the second point may be based on end tidal CO2 (PetCO2). The physiological measurements for the second point may be captured at the end of a rest phase of a CPX test.

A midpoint of the vector may be identified between the first point and the second point. The midpoint may be mapped to a zone. For example, the zones may correspond to quadrants of a graph. The quadrants may be divided based on PECO2 and PetCO2 values. For example, in some implementations, a quadrant corresponding to a transitional zone may be defined as having a PECO2 value of less than 25 mmHg and a PetCO2 value of less than 35 mmHg; a quadrant corresponding to a left ventricular (LV) dysfunction zone may be defined as having a PECO2 value equal to or greater than 25 mmHg and a PetCO2 value of less than 35 mmHg; a quadrant corresponding to a chronic obstructive pulmonary disease (COPD)/restrictive lung disease (RLD) zone may be defined as having a PECO2 value of less than 25 mmHg and a PetCO2 value equal to or greater than 35 mmHg; and a quadrant corresponding to normal physiology may be defined as having a PECO2 value equal to or greater than 25 mmHg and a PetCO2 value equal to or greater than 35 mmHg.

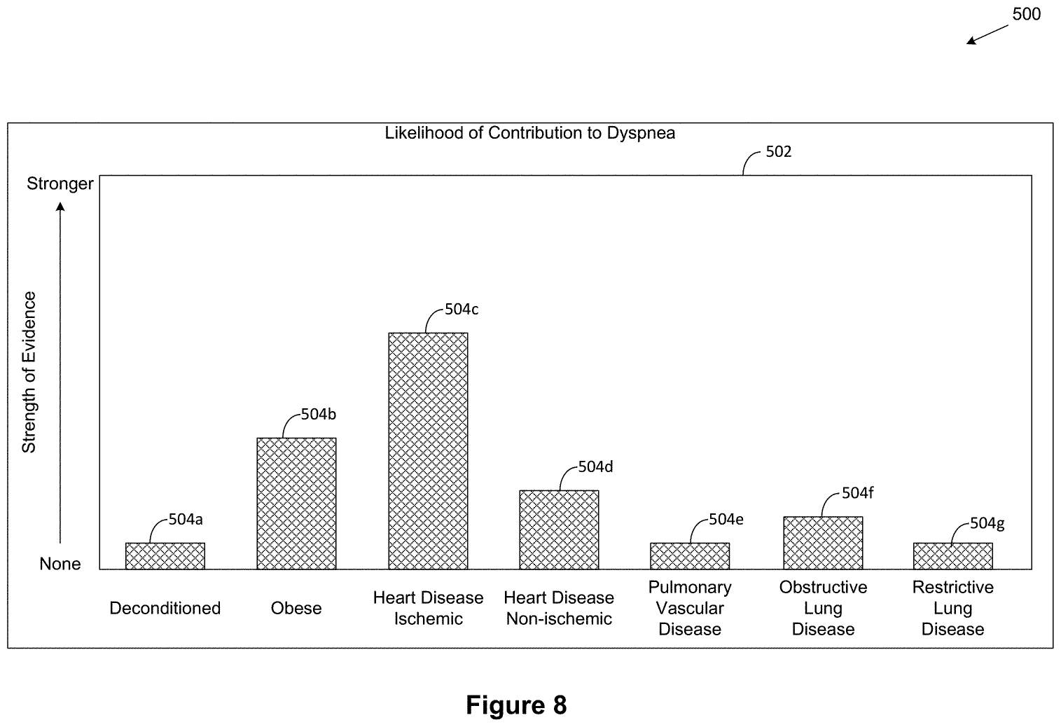

Step 6—Use this total SII Score to set the silo height for the Ischemic Heart Disease silo in a plot as shown in FIG. 8.

FIG. 8 illustrates an example user interface 500 that may be generated by embodiments of the system 100. The user interface 500 includes a contribution graph 502. In some embodiments, the user interface is generated by a user interface engine that may be implemented as instructions stored in the memory 112 or the secondary storage device 122.

The contribution graph 502 includes one or more silos 504a, 504b, 504c, 504d, 504e, 504f, and 504g (referred to collectively as silos 504). The silos 504 correspond to the contribution of a particular disease or condition to dyspnea of the patient. In the example shown, there are silos for Deconditioned, Obese, Heart Disease Ischemic, Heart Disease Non-Ischemic, Pulmonary Vascular Disease, Obstructive Lung Disease, and Restrictive Lung Disease. In some embodiments, the height of the silos is associated with the degree of contribution to dyspnea calculated for the disease or condition. The height of the heart disease ischemic silo 504c may be calculated in accordance with the logic of FIG. 7. Methods for calculating the heights of other silos are described further in U.S. Pat. No. 10,010,264, titled “Pattern recognition system for quantifying the likelihood of the contribution of multiple possible forms of chronic disease to patient reported dyspnea” and dated Jul. 3, 2018.

Some implementations recommend therapeutic interventions (treatments) based on the SII value. Recommending a treatment may include generating interpretation remarks. The interpretation remarks may be presented for review and revision by a care provider such as a medical doctor.

For example, if the SII value is 2 or higher, some implementations may recommend consideration of therapy with a vasodilator or calcium channel blocker. In some implementations, the recommended therapeutical interventions may include implantation of a medical device such as a stent or bypass surgery. Some implementations may re-evaluate a patient after a therapeutic intervention and may generate interpretation remarks based on any changes in the SII value between CPX tests performed before and after the therapeutic intervention.

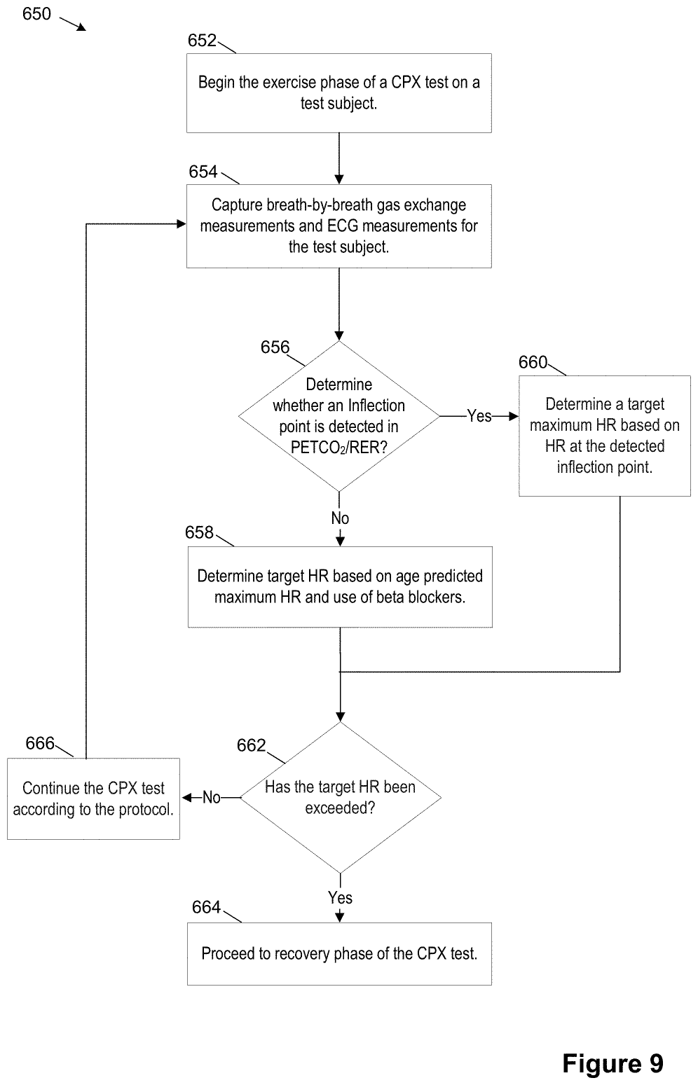

FIG. 9 is a flowchart of an example method 650 for administering a CPX test. Test administrators of a CPX test, for purposes of cardiac ischemia detection, may not know when to stop patient exercise (other than obtaining a “quit” signal from the patient) and proceed to the recovery phase of the procedure. The method 650 uses exercise gas exchange and ECG analysis to more accurately assess patient max or near max effort to determine when to stop the exercise phase of a CPX test. This method 650 may be used in addition to relative dynamic observance of adverse ST segment changes and potential arrhythmias.

Some implementations guide the CPX test for ischemia detection by CPX measures (or measurements or variables) that indicate the test subject is beyond their anaerobic threshold, where EF usually peaks, and approaching upper limits of tolerable exertion. Typically, the target heart rate for a successful 12 lead ECG treadmill stress test is 85% of the test subject's max predicted heart rate or 75% if beta blocker medication is being administered.

The monitoring of a combined gas exchange-based metric, indicative of blood flow and test subject effort, is a useful non-invasive “tool” to determine the test point where the test subject can proceed to recovery for additional ECG monitoring of ST changes and heart rate decay.

The method 650 may be used for dynamic, real time monitoring of a cycle ergometer test and would employ a means to visually track the ratio of PETCO₂ divided by RER. When the RER starts to steadily increase, reflecting the approach of more anaerobically supported skeletal muscle metabolism, together with a decrease in PETCO₂ typically occurring at the VT where stroke volume and pulmonary blood flow start to decrease in their rate of rise, an abrupt decrease is amplified in the PETCO₂/RER ratio curve. Depending also on the following observance of significant ST depression changes in the various limb and pre-cordial ECG leads >0.1 mv or 1 mm, a decision can then be made regarding whether the test subject should continue exercising for a period of time prior to advancing to the recovery stage of the test.

In at least some implementations, the method 650 may assure that an adequate level of safe stress is performed by the test subject. Beneficially, the method 650 may rely more on dynamic metabolic test subject performance rather than target HR or ST segment values alone.

At operation 652, an exercise phase of a CPX test is started on a test subject. As described previously, the exercise phase may follow a rest phase and possibly a warm-up (e.g., a free-wheel) phase. In some implementations, the CPX test includes the test subject exercising on a cycle ergometer, hand ergometer, treadmill, or step.

At operation 654, breath-by-breath gas exchange measurements are captured for the test subject. The breath-by-breath gas exchange measurements may be captured using, for example, the system 100.

At operation 656, it is determined whether an inflection point is detected in the PETCO₂/RER ratio. In some implementations, the inflection point is detected using the method 400. In some implementations, the inflection point is detected based on breath-by-breath measurements as they are captured based on a downward deflection of the PETCO₂/RER ratio. The downward deflection may be identified by, for example, a negative derivative of the PETCO₂/RER ratio. An example of an inflection point is shown at 210 in FIG. 2. If the inflection point is not detected, the method continues to operation 658. If the inflection point is detected, the method proceeds to operation 660.

At operation 658, a target HR is determined based on an age-predicted maximum HR for the test subject and whether the test subject has been medicated with beta blockers. For example, the target HR may be determined to be 85% of the age-predicted maximum heart rate if the test subject has not been medicated with beta blockers or 75% of the age-predicted maximum HR if the test subject has been medicated with beta blockers.

If the method continues to operation 660, the target heart rate is instead determined based on the HR at the inflection point detected in operation 656. For example, the target HR may be 10% greater than the HR determined at the detected inflection point.

At operation 662, it is determined whether the target HR (e.g., as determined at operations 658 or 660) has been exceeded. If the target HR has been exceeded, the method continues to operation 664. If not, the method continues to operation 666 where the test continues according to the test protocol and returns to operation 654.

At operation 664, the test proceeds to a recovery phase. In some implementations, the system 100 automatically proceeds to the recovery phase. In some implementations, the system 100 may prompt a test administrator to proceed to the recovery phase. In some implementations, the system 100 will continue the exercise phase for a predetermined duration of time (e.g., 1 minute, 2 minutes, 3 minutes) after the target HR is exceeded before proceeding to the recovery phase.

It should be understood that if the test subject experiences chest pressure or pain, the test may immediately proceed, regardless of the target HR, to the recovery phase for continued monitoring of potential ST segment depression or elevation.

Some clinical advantages of embodiments of the method 650 include that the 1) the ST segment of the ECG, 2) select CPET metrics representing the level of patient exertion, and 3) a % of the target heart rate can all be viewed on a test data monitoring screen with dynamic increase in cycle ergometer watts. This may enhance test subject safety during the test and focus testing on a zone of exertion where cardiac ischemia is more apt to occur relative to daily activities.

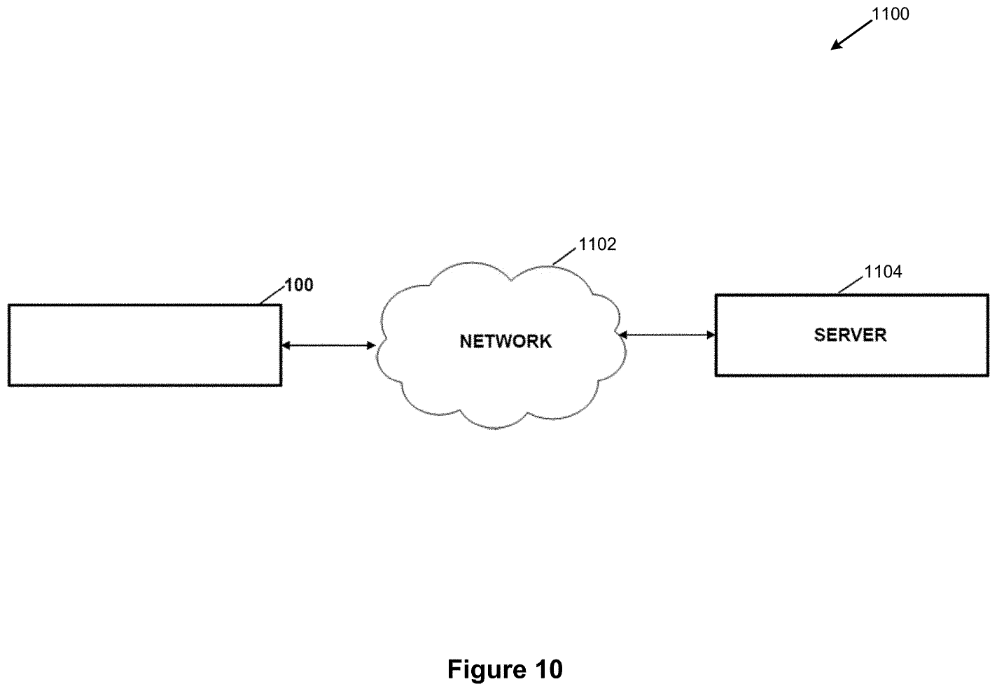

Referring now to FIG. 10, an example networked CPX testing system 1100 is illustrated. The networked CPX testing system 1100 can be used for remote testing and monitoring of patients. For example, the networked CPX testing system 1100 can be used in non-clinical environments, such as at the patient's home. In this example, the networked CPX testing system 1100 includes the CPX testing system 100, network 1102, and server 1104.

In some embodiments, the CPX testing system 100 is configured to send data associated with gas exchange tests (such as measurements of physiological variables (sometimes referred to herein as physiological measurements), index scores, etc.) to the server 1104 over the network 1102.

The network 1102 is an electronic communication network that facilitates communication between the CPX testing system 100 and the server 1104. An electronic communication network is a set of computing devices and links between the computing devices. The computing devices in the network use the links to enable communication among the computing devices in the network. The network 1102 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network 1102 includes various types of links. For example, the network 1102 can include wired and/or wireless links, including Bluetooth, ultra-wideband (UWB), 802.11, ZigBee, and other types of wireless links. Furthermore, in various embodiments, the network 1102 is implemented at various scales. For example, the network 1102 can be implemented as one or more local area networks (LANs), metropolitan area networks, subnets, wide area networks (such as the Internet), or can be implemented at another scale.

The server 1104 comprises one or more computing devices. Various embodiments of computing devices have been described above. Further, in some embodiments, the server 1104 comprises a single server or a bank of servers. In another example, the server 1104 can be a distributed network server, commonly referred to as a “cloud” server.

In some embodiments, the server 1104 operates to receive data such as test results and physiological measurements from the CPX testing system 100. The server 1104 can then process the data and store it in one or more of a database or electronic medical records system.

In some embodiments, the server 1104 generates user interfaces, such as with a user interface engine, and transmits those user interfaces for display remotely. For example, the server 1104 may generate a web page comprising a user interface containing test data transmitted from the CPX testing system 100. The web page may then be transmitted to a computing device (e.g., a smartphone, personal computer, or tablet) of the patient or a caregiver.

Additionally, in some embodiments, the CPX testing system 100 communicates with a cellular phone or other network-connected computing device to access the network 1102. For example, the CPX testing system 100 may transmit data to the server 1104 via communication with a cell phone using Bluetooth. Other embodiments are possible as well.

This embodiments disclosed herein may be used in a scoring system based on several CPX test variables that are used to determine the height of “disease silos” graphic representations of the likeliness that the cause of a patient's dyspnea is one or more of the represented the disease silos. For example, embodiments may be incorporated into a system for determining and displaying “disease silos” such as those described in U.S. Pat. No. 10,010,264, titled “Pattern recognition system for quantifying the likelihood of the contribution of multiple possible forms of chronic disease to patient reported dyspnea” and dated Jul. 3, 2018. This allows a non-expert physician to emulate the thought process of experienced physicians and physiologists to determine the primary and secondary causes of the patient's shortness of breath. The methods described herein may improve the likeliness scoring by expanding the individual silo scoring schemes to include the Ventilation/Perfusion measurements of the present method to the scoring algorithms disclosed in in U.S. Pat. No. 10,010,264.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

In the following several examples are given.

Example 1: A system comprising: a flow sensor configured to sense a respiratory flow of the patient; an analyzer configured to determine a respiratory gas composition of at least a portion of the respiratory flow of the patient; and a computing device configured to: receive gas exchange measurements, wherein the gas exchange measurements are based on breath-by-breath data captured by the flow sensor and the analyzer during a cardiopulmonary exercise test that includes an exercise phase; determine an ischemic index value based on the received gas exchange measurements; and output the ischemic index value. The ischemic index value may indicate the strength of evidence that the patient is suffering from ischemic heart disease.

Example 2: The system of example 1 further comprising an ECG device configured to determine an ST segment value for the patient, wherein the ST segment values are captured by the ECG device during the cardiopulmonary exercise test; and the ischemic index value is determined based on the received gas exchange measurements and ST segment values.

Example 3: The system of example 1, wherein the ST segment values include ST segment depression values and wherein the ischemic index value is determined based on the received gas exchange measurements and ST segment depression values. The ST segment values may also include ST segment elevation values and the ischemic index value may be determined based on the received gas exchange measurements and ST segment depression and elevation values.

Example 4: The system of example 3, wherein the ischemic index value is determined based on an ST segment depression occurring during the exercise phase and continuing through a recovery phase.

Example 5: The system of example 1, wherein the computing device is further configured to: determine a heart rate threshold for the patient; receive heart rate measurements for the patient during the exercise phase; compare the heart rate measurements to the heart rate threshold; and responsive to determining the heart rate measurements exceed the heart rate threshold, alter the exercise phase. The heart rate measurements may be captured with the ECG device. In some implementations, the system also includes a pulse oximetry device and the heart rate measurements are captured with the pulse oximetry device. The heart rate threshold for the patient may be determined based on an age of the patient. The heart rate threshold may also be determined based on whether the patient has been administered beta blocker medication.

Example 6: The system of example 1, wherein the ischemic index value indicates a likelihood that the patient suffers from cardiac ischemia.

Example 7: The system of examples 1 or 2, wherein the computing device being configured to determine an ischemic index value based on the received gas exchange measurements includes the computing device being configured to determine whether a breakpoint occurs within a plot of at least one physiological measurement against work rate during the exercise phase of the cardiopulmonary exercise test.

Example 8: The system of example 7, wherein the at least one physiological measurement includes heart rate, VO2, or GxCAP.

Example 9: The system of example 7, wherein the computing device being configured to determine whether a breakpoint occurs within a plot of at least one physiological measurement against work rate during the exercise phase of the cardiopulmonary exercise test includes the computing device being configured to: plot a plurality of data points for the at least one physiological measurement and work rate; identify a middle portion of the plot based on comparing the at least one physiological measurement to an upper threshold value; fit a first line to the identified middle portion of the plot; identify an upper portion of the plot based on comparing the physiological measurement value of the data points to the upper threshold value; and determine whether at least one data point in the identified upper portion has a physiological measurement value that exceeds the first line by at least a deviation threshold.

Example 10: The system of examples 1 or 2, wherein the computing device being configured to determine an ischemic index value based on the received gas exchange measurements includes the computing device being configured to: determine a plurality of breakpoint scores, each of the breakpoint scores being associated with a different physiological measurement and being based on whether a breakpoint occurs within a plot of the associated physiological measurement against work rate during the exercise phase of the cardiopulmonary exercise test; and combine the plurality of breakpoint scores.

Example 11: The system of example 10, wherein the computing device being configured to combine the plurality of breakpoint scores includes the computing device being configured to average the plurality of breakpoint scores.

Example 12: The system of example 10, wherein the computing device being configured to combine the plurality of breakpoint scores includes the computing device being configured to sum the plurality of breakpoint scores and scale the summed breakpoint scores.

Example 13: The system of examples 1 or 2, wherein the ischemic index value is further determined based on a midpoint of a ventilation/perfusion vector plotted from the gas exchange measurements, wherein the vector includes a first point based on a rest value and a second point based on an exercise value, a first coordinate value of the first point and a first coordinate value of the second point being based on mixed expired CO₂ (PECO₂) and a second coordinate value of the first point and a second coordinate value of the second point being based on end tidal CO₂ (PetCO₂).

Example 14: The system of example 13, wherein the ischemic index value is increased if the midpoint of the vector corresponding to a LV dysfunction or transitional zone of a ventilation/perfusion plot.

Example 15: The system of examples 1 or 2, wherein the computing device being configured to output the ischemic index includes the computing device being configured to display a graph that includes an indicator representing ischemic heart disease, a visual property of the indicator representing ischemic heart disease being based on the ischemic index. The graph may also include one or more additional indicators representing other physiological conditions. Examples of other physiological conditions include deconditioned, obsess, heart disease (non-ischemic), pulmonary vascular disease, obstructive lung disease, and restrictive lung disease.

Example 16: The system of example 2, wherein the computing device is further configured to output a graph based on one or more traces from leads of the ECG device, wherein the graph includes a region that is shaded based on ST segment elevation/depression of an associated lead of the ECG device.

Example 17: A system comprising: a flow sensor configured to sense a respiratory flow of the patient; an analyzer configured to determine a respiratory gas composition of at least a portion of the respiratory flow of the patient; an ECG device configured to determine an ST segment value for the patient; and a computing device configured to: receive gas exchange measurements, wherein the gas exchange measurements are based on breath-by-breath data captured by the flow sensor and the analyzer during a cardiopulmonary exercise test that includes an exercise phase; receive ST segment values, wherein the ST segment values are captured by the ECG device during the cardiopulmonary exercise test; and output a visual representation of the gas exchange measurements and the ST segment values, the visual representation including regions that are shaded based on ST segment elevation/depression of the ST segment value.

Example 18: The system of example 17, wherein the visual representation includes at least one row corresponding to a lead of the ECG device, the row being shaded to indicate ST segment elevation/depression at different times during the cardiopulmonary test, and the visual representation including a plot based of a physiological value determined from the gas exchange measurements.

Example 19: The system of example 18, wherein a portion of the at least one row is highlighted based on a time during the cardiopulmonary test at which a breakpoint was detected based on the gas exchange measurements.

Example 20: A system comprising: a flow sensor configured to sense a respiratory flow of the patient; an analyzer configured to determine a respiratory gas composition of at least a portion of the respiratory flow of the patient; an ECG device configured to determine an ST segment value for the patient; and a computing device configured to: receive gas exchange measurements, wherein the gas exchange measurements are based on breath-by-breath data captured by the flow sensor and the analyzer during a cardiopulmonary exercise test that includes an exercise phase; receive ST segment values, wherein the ST segment values are captured by the ECG device during the cardiopulmonary exercise test; determine an ischemic index value based on the received gas exchange measurements and ST segment values; output the ischemic index; and output a visual representation of the gas exchange measurements and the ST segment values, the visual representation including regions that are shaded based on ST segment elevation/depression of the ST segment value.

Example 21: A method comprising: receiving gas exchange measurements, wherein the gas exchange measurements are based on breath-by-breath data captured by the flow sensor and the analyzer during a cardiopulmonary exercise test that includes an exercise phase; receiving ST segment values, wherein the ST segment values are captured by the ECG device during the cardiopulmonary exercise test; determining an ischemic index value based on the received gas exchange measurements and ST segment values; and outputting the ischemic index value.

Example 22: The method of example 21, further comprising: capturing the gas exchange measurements with a flow sensor configured to sense a respiratory flow of the patient and an analyzer configured to determine a composition of at least a portion of the respiratory flow of the patient; and determining ST segment values for the patient with an ECG device.

Example 23: The method of example 21, wherein the ST segment values include ST segment depression values and the ischemic index value is determined based on the received gas exchange measurements and ST segment depression values.

Example 24: The method of example 23, wherein the ST segment values include ST segment elevation values.

Example 25: The method of example 24, wherein the ischemic index value is determined based on the received gas exchange measurements and ST segment depression and elevation values.

Example 26: The method of example 21, wherein the ischemic index value indicates a likelihood that the patient suffers from cardiac ischemia.

Example 27: The method of example 21, wherein the determining an ischemic index value based on the received gas exchange measurements and ST segment values includes determining whether a breakpoint occurs within a plot of at least one physiological measurement against work rate during the exercise phase of the cardiopulmonary exercise test.

Example 28: The method of example 27, wherein the at least one physiological measurement includes heart rate, VO2, or GxCAP.

Example 29: The method of example 27, wherein the determining whether a breakpoint occurs within a plot of at least one physiological measurement against work rate during the exercise phase of the cardiopulmonary exercise test includes: plotting a plurality of data points for the at least one physiological measurement and work rate; identifying a middle portion of the plot based on comparing the at least one physiological measurement to an upper threshold value; fitting a first line to the identified middle portion of the plot; identifying an upper portion of the plot based on comparing the physiological measurement value of the data points to the upper threshold value; and determining whether at least one data point in the identified upper portion has a physiological measurement value that exceeds the first line by at least a deviation threshold.

Example 30: The method of example 21, wherein the determining an ischemic index value based on the received gas exchange measurements and ST segment values includes: determining a plurality of breakpoint scores, each of the breakpoint scores being associated with a different physiological measurement and being based on whether a breakpoint occurs within a plot of the associated physiological measurement against work rate during the exercise phase of the cardiopulmonary exercise test; and combining the plurality of breakpoint scores.

Example 31: The method of example 30, wherein the combining the plurality of breakpoint scores includes the computing device being configured to average the plurality of breakpoint scores.

Example 32: The method of example 30, wherein the combining the plurality of breakpoint scores includes the summing the plurality of breakpoint scores and scaling the summed breakpoint scores.

Example 33: The method of example 21, wherein the ischemic index value is further determined based on a midpoint of a ventilation/perfusion vector plotted from the gas exchange measurements, wherein the vector includes a first point based on a rest value and a second point based on an exercise value, a first coordinate value of the first point and a first coordinate value of the second point being based on mixed expired CO2 (PECO2) and a second coordinate value of the first point and a second coordinate value of the second point being based on end tidal CO2 (PetCO2).

Example 34: The method of example 33, wherein the ischemic index value is increased if the midpoint of the vector corresponds to a LV dysfunction or transitional zone of a ventilation/perfusion plot.

Example 35: The method of example 1, wherein the outputting the ischemic index includes displaying a graph that includes an indicator representing ischemic heart disease, a visual property of the indicator representing ischemic heart disease being based on the ischemic index value.

Example 36: The method of example 21, further comprising outputting a graph based on one or more traces from leads of the ECG device, wherein the graph includes a region that is shaded based on ST segment elevation/depression of an associated lead of the ECG device.

Example 37: A method comprising: receiving gas exchange measurements, wherein the gas exchange measurements are based on breath-by-breath data captured by a flow sensor and an analyzer during a cardiopulmonary exercise test that includes an exercise phase; receiving ST segment values, wherein the ST segment values are captured by an ECG device during the cardiopulmonary exercise test; and outputting a visual representation of the gas exchange measurements and the ST segment values, the visual representation including regions that are shaded based on ST segment elevation/depression of the ST segment value.

Example 38: The method of example 37 wherein the visual representation includes at least one row corresponding to a lead of the ECG device, the row being shaded to indicate ST segment elevation/depression at different times during the cardiopulmonary test, and the visual representation including a plot based of a physiological value determined from the gas exchange measurements.

Example 39: The method of example 38, wherein a portion of the at least one row is highlighted based on a time during the cardiopulmonary test at which a breakpoint was detected based on the gas exchange measurements.

Example 40: A non-transitory computer-readable storage medium comprising instructions stored thereon that, when executed by at least one processor, cause a computing method to perform the method of any of examples 21-39.

What is claimed is:

1. A system comprising:
a flow sensor configured to sense a respiratory flow of a patient;
an analyzer configured to determine a respiratory gas composition of at least a portion of the respiratory flow of the patient;
an ECG device configured to determine an ST segment value for the patient; and
a computing device configured to:
receive gas exchange measurements, wherein the gas exchange measurements are based on breath-by-breath data captured by the flow sensor and the analyzer during a cardiopulmonary exercise test that includes an exercise phase;
receive ST segment values, wherein the ST segment values are captured by the ECG device during the cardiopulmonary exercise test;
determine an ischemic index value based on the received gas exchange measurements and ST segment values; and
output the ischemic index value.

2. The system of claim 1, wherein the ST segment values include ST segment depression values and the ischemic index value is determined based on the received gas exchange measurements and ST segment depression values.

3. The system of claim 2, wherein the ST segment values include ST segment elevation values and wherein the ischemic index value is determined based on the received gas exchange measurements and ST segment depression and elevation values.

4. The system of claim 1, wherein the ischemic index value indicates a likelihood that the patient suffers from cardiac ischemia.

5. The system of claim 1, wherein the computing device is further configured to:
determine a heart rate threshold for the patient;
receive heart rate measurements for the patient during the exercise phase;
compare the heart rate measurements to the heart rate threshold; and
responsive to determining the heart rate measurements exceed the heart rate threshold, alter the exercise phase.

6. The system of claim 5, wherein the heart rate threshold for the patient is determined based on an age of the patient.

7. The system of claim 1, wherein the computing device being configured to determine an ischemic index value based on the received gas exchange measurements and ST segment values includes the computing device being configured to determine whether a breakpoint occurs within a plot of at least one physiological measurement against work rate during the exercise phase of the cardiopulmonary exercise test.

8. The system of claim 7, wherein the at least one physiological measurement includes heart rate, VO2, or GxCAP.

9. The system of claim 7, wherein the computing device being configured to determine whether a breakpoint occurs within a plot of at least one physiological measurement against work rate during the exercise phase of the cardiopulmonary exercise test includes the computing device being configured to:
plot a plurality of data points for the at least one physiological measurement and work rate;
identify a middle portion of the plot based on comparing the at least one physiological measurement to an upper threshold value;
fit a first line to the identified middle portion of the plot;
identify an upper portion of the plot based on comparing the physiological measurement value of the data points to the upper threshold value; and
determine whether at least one data point in the identified upper portion has a physiological measurement value that exceeds the first line by at least a deviation threshold.

10. The system of claim 1, wherein the computing device being configured to determine an ischemic index value based on the received gas exchange measurements and ST segment values includes the computing device being configured to:
determine a plurality of breakpoint scores, each of the breakpoint scores being associated with a different physiological measurement and being based on whether a breakpoint occurs within a plot of the associated physiological measurement against work rate during the exercise phase of the cardiopulmonary exercise test; and
combine the plurality of breakpoint scores.

11. The system of claim 10, wherein the computing device being configured to combine the plurality of breakpoint scores includes the computing device being configured to average the plurality of breakpoint scores.

12. The system of claim 10, wherein the computing device being configured to combine the plurality of breakpoint scores includes the computing device being configured to sum the plurality of breakpoint scores and scale the summed breakpoint scores.

13. The system of claim 1, wherein the ischemic index value is further determined based on a midpoint of a ventilation/perfusion vector plotted from the gas exchange measurements, wherein the vector includes a first point based on a rest value and a second point based on an exercise value, a first coordinate value of the first point and a first coordinate value of the second point being based on mixed expired CO₂ (PECO₂) and a second coordinate value of the first point and a second coordinate value of the second point being based on end tidal CO₂ (PetCO₂).

14. The system of claim 13, wherein the ischemic index value is increased if the midpoint of the vector corresponds to a LV dysfunction or transitional zone of a ventilation/perfusion plot.

15. The system of claim 1, wherein the computing device being configured to output the ischemic index includes the computing device being configured to display a graph that includes an indicator representing ischemic heart disease, a visual property of the indicator representing ischemic heart disease being based on the ischemic index value.

16. The system of claim 1, wherein the computing device is further configured to output a graph based on one or more traces from leads of the ECG device, wherein the graph includes a region that is shaded based on ST segment elevation/depression of an associated lead of the ECG device.

17. A system comprising:
a flow sensor configured to sense a respiratory flow of a patient;
an analyzer configured to determine a respiratory gas composition of at least a portion of the respiratory flow of the patient;
an ECG device configured to determine an ST segment value for the patient; and
a computing device configured to:
receive gas exchange measurements, wherein the gas exchange measurements are based on breath-by-breath data captured by the flow sensor and the analyzer during a cardiopulmonary exercise test that includes an exercise phase;
receive ST segment values, wherein the ST segment values are captured by the ECG device during the cardiopulmonary exercise test; and
output a visual representation of the gas exchange measurements and the ST segment values, the visual representation including regions that are shaded based on ST segment elevation/depression of the ST segment value.

18. The system of claim 17, wherein the visual representation includes at least one row corresponding to a lead of the ECG device, the row being shaded to indicate ST segment elevation/depression at different times during the cardiopulmonary test, and the visual representation including a plot based of a physiological value determined from the gas exchange measurements.

19. The system of claim 18, wherein a portion of the at least one row is highlighted based on a time during the cardiopulmonary test at which a breakpoint was detected based on the gas exchange measurements.

20. A system comprising:
a flow sensor configured to sense a respiratory flow of a patient;
an analyzer configured to determine a respiratory gas composition of at least a portion of the respiratory flow of the patient;
an ECG device configured to determine an ST segment value for the patient; and
a computing device configured to:
receive gas exchange measurements, wherein the gas exchange measurements are based on breath-by-breath data captured by the flow sensor and the analyzer during a cardiopulmonary exercise test that includes an exercise phase;
receive ST segment values, wherein the ST segment values are captured by the ECG device during the cardiopulmonary exercise test;
determine an ischemic index value based on the received gas exchange measurements and ST segment values;
output the ischemic index; and
output a visual representation of the gas exchange measurements and the ST segment values, the visual representation including regions that are shaded based on ST segment elevation/depression of the ST segment value.
